(19) United States
(12) Patent Application Publication
(10) Pub. No.: US 2022/0233642 A1
(43) Pub. Date: Jul. 28, 2022

(54) TREATMENT OF ARDS

(71) Applicant: THE UNIVERSITY COURT OF THE UNIVERSITY OF EDINBURGH, Edinburgh Midlothian (GB)

(72) Inventors: Sarah Walmsley, Edinburgh Lothian (GB); Moira Whyte, Edinburgh Lothian (GB); Stuart Forbes, Edinburgh Lothian (GB); Ananda Mirchandani Thanwerdas, Edinburgh Lothian (GB); Stephen Jenkins, Edinburgh Lothian (GB)

(21) Appl. No.: 17/610,296

(22) PCT Filed: May 15, 2020
(86) PCT No.: PCT/GB2020/051184

§ 371 Date: Nov. 10, 2021

(30) Foreign Application Priority Data
May 17, 2019 (GB) 1906975.6

(51) Int. Cl. A61K 38/19; A61P 29/00; A61P 11/00; A61K 35/15; A61K 38/20

(52) U.S. Cl. CPC A61K 38/193; A61P 29/00; A61K 38/20; A61K 35/15; A61P 11/00

(57) ABSTRACT

The present invention relates to an agent for increasing mononuclear cell number, and/or inducing a pro-restorative phenotype in mononuclear cells for use in the treatment of a lung disease associated with dysfunctional neutrophilic inflammation, particularly wherein the agent is CSF1 and the lung disease is acute respiratory distress syndrome (ARDS).

### FIELD OF INVENTION

The present invention relates to an agent for use in the treatment of a lung disease associated with dysfunctional neutrophilic inflammation. The invention also relates to methods of treating a lung disease associated with dysfunctional neutrophilic inflammation, as well as uses of an agent for increasing mononuclear cell number, and/or inducing a pro-restorative phenotype in mononuclear cells, in the manufacture of a medicament for the treatment of a lung disease associated with dysfunctional neutrophilic inflammation.

### BACKGROUND

Acute respiratory distress syndrome (ARDS) is a life-threatening condition. It is usually a complication of a serious existing condition, such as pneumonia, septicaemia, severe flu, or major trauma. ARDS has a substantial mortality rate and currently no effective treatments exist.

ARDS is caused by an acute inflammatory response in the lungs which leads to hypoxia. It is believed that hypoxic conditions alter the function and survival of neutrophils resulting in a hyperinflammatory response that is damaging (Walmsley, 2005; Eltzschig 2011). In ARDS, there is an accumulation of dysfunctional neutrophils in the lungs. This is one of the hallmarks of ARDS (Zemans and Matthay 2016).

Mononuclear cells are a class of cells that are found throughout the body and are responsible for phagocytosis of bacteria, viruses, and other foreign substances, as well as abnormal body cells including neutrophils that have undergone apoptosis.

Mononuclear cells are derived from precursor cells in the bone marrow. These precursors develop into monocytes and dendritic cells, phagocytic cells that are released into the bloodstream. Some monocytes and dendritic cells remain in the general blood circulation, but most of them enter body tissues. In tissues, monocytes develop into much larger phagocytic cells known as macrophages. The great majority of macrophages remain as stationary cells within tissue, where they filter out and destroy foreign particles.

Wang et al. (2018) have suggested that that M2-polarised bone marrow-derived macrophages may have a protective effect in ARDS by reducing neutrophil infiltration. However, treatment approaches which reduce the number of neutrophils and/or clear neutrophils from the lungs are highly desirable. To date no such successful treatments have been described.

The present invention aims to overcome or ameliorate the problems associated with the prior art.

### SUMMARY OF THE INVENTION

In one aspect the invention provides an agent for increasing mononuclear cell number in a subject for use in the treatment of a lung disease associated with dysfunctional neutrophilic inflammation.

In a further aspect, the invention provides a method for treating a lung disease associated with dysfunctional neutrophilic inflammation in a subject, the method comprising providing the subject with a therapeutically effective amount of an agent for increasing mononuclear cell number in the subject.

In a further aspect, the invention provides use of an agent for increasing mononuclear cell number in the manufacture of a medicament for the treatment of a lung disease associated with dysfunctional neutrophilic inflammation.

Suitably, the agent may increase monocyte and/or macrophage cell number in the lungs of the subject.

Suitably the agent may increase the pro-restorative mononuclear cell number in a subject, suitably the agent may increase the number of mononuclear cells with a pro-restorative phenotype in a subject. Suitably the agent may increase the pro-restorative monocyte and/or pro-restorative macrophage cell number in a subject, suitably the agent may increase the number of monocytes with a pro-restorative phenotype in a subject and/or increase the number of macrophages with a pro-restorative phenotype in a subject. Suitably, the agent may increase the number of monocytes with a pro-restorative phenotype, which in turn increases the number of macrophages with a pro-restorative phenotype.

Alternatively or additionally, the agent may induce a pro-restorative phenotype in the mononuclear cell population in a subject. Suitably the pro-restorative phenotype may be induced together with an increase in the overall number of mononuclear cells in a subject. Alternatively, the pro-restorative phenotype may be induced without increasing the overall number of mononuclear cells in a subject, but with an increase in the proportion of mononuclear cells of a subject that have a pro-restorative phenotype.

In a further aspect, the invention provides an agent for inducing a pro-restorative phenotype in mononuclear cells in a subject for use in the treatment of lung disease associated with dysfunctional neutrophilic inflammation.

In a further aspect, the invention provides a method for treating a lung disease associated with dysfunctional neutrophilic inflammation in a subject, the method comprising providing the subject with a therapeutically effective amount of an agent for inducing a pro-restorative phenotype in mononuclear cells in the subject.

In a further aspect, the invention provides use of an agent for inducing a pro-restorative phenotype in mononuclear cells in the manufacture of a medicament for the treatment of a lung disease associated with dysfunctional neutrophilic inflammation.

Suitably the pro-restorative phenotype may be induced in monocytes and/or macrophages. In one embodiment, the pro-restorative phenotype is induced in monocytes. Suitably the pro-restorative monocytes then become pro-restorative macrophages.

Suitably, the agent may be selected from the group consisting of a polypeptide, or a fragment, variant or homolog thereof; a nucleic acid; a mononuclear cell; a small molecule; or any combination thereof. Suitably, the polypeptide may be selected from the group consisting of a cytokine, an enzyme, a hormone, a growth factor, a regulatory protein, and an immunoregulator. Suitably, the cytokine may be selected from the group consisting of CSF1 and IL-34. More suitably, the agent may be CSF1. Such an embodiment gives rise to several aspects of the present invention.

Suitably, CSF1 may have an amino acid sequence at least 75% identical to SEQ ID NO: 1.

Suitably, the nucleic acid may be DNA or RNA. Suitably, the nucleic acid may be an expression vector comprising a sequence encoding a polypeptide, such as a polypeptide encoding CSF1, or a fragment, variant or homolog thereof.

Suitably, the agent may be mononuclear cells. Suitably, the mononuclear cells may be selected from any one or more of: a monocyte, a macrophage, or a monocyte precursor. Suitably the mononuclear cells may be mononuclear cells having a pro-restorative phenotype. Suitably the mononuclear cells may be pro-restorative mononuclear cells. Suitably, the monocyte may be may be a circulatory monocyte or a tissue monocyte (for example a lung monocyte), have one or more markers selected from the group consisting of CD45⁺, HLADR⁺, CD14⁺, CD16⁺, CD11b⁺, CD206⁻, CD169⁻ and/or be non-granulocytic.

Suitably, the macrophage may be a monocyte derived macrophage and/or tissue macrophage. The monocyte derived macrophage and/or tissue macrophage may be a lung macrophage. The lung macrophage may be an alveolar macrophage or an interstitial macrophage. The monocyte derived macrophage may have one or more markers characteristic for an alveolar macrophage or an interstitial macrophage. Suitably, the monocyte derived macrophage may have one or more markers selected from the group consisting of CD11b⁺, HLADR⁺, CD206⁺, and CD169⁺. Optionally, the alveolar macrophage may be CD15- or may have one or more markers selected from the group consisting of CD11b⁺, HLA-DR⁺, CD206⁺, CD169⁻. Optionally, the interstitial macrophage may be CD15⁻. Alternatively, the macrophage may be a bone marrow derived macrophage.

In a further aspect, the invention provides Colony Stimulating Factor 1 (CSF1), or a fragment, variant or homolog thereof, for use in the treatment of a lung disease associated with dysfunctional neutrophilic inflammation.

In a further aspect, the invention provides a method for treating a lung disease associated with dysfunctional neutrophilic inflammation in a subject, the method comprising providing the subject with a therapeutically effective amount of Colony Stimulating Factor 1 (CSF1), or a fragment, variant or homolog thereof.

In a further aspect, the invention provides use of Colony Stimulating Factor 1 (CSF1), or a fragment, variant or homolog thereof, in the manufacture of a medicament for the treatment of a lung disease associated with a dysfunctional neutrophilic inflammation.

In a further aspect, the invention provides a mononuclear cell having a pro-restorative phenotype for use in the treatment of a lung disease associated with dysfunctional neutrophilic inflammation.

In a further aspect, the invention provides a method for treating a lung disease associated with dysfunctional neutrophilic inflammation in a subject, the method comprising providing the subject with a therapeutically effective amount of a mononuclear cell having a pro-restorative phenotype.

In a further aspect, the invention provides use of a mononuclear cell having a pro-restorative phenotype in the manufacture of a medicament for the treatment of a lung disease associated with a dysfunctional neutrophilic inflammation.

In all aspects of the present invention, the lung disease associated with dysfunctional neutrophilic inflammation may be selected from the group consisting of acute respiratory distress syndrome (ARDS), chronic obstructive pulmonary disease (COPD) and lung infection.

Except for where the context requires otherwise, the considerations set out in this disclosure should be considered to be applicable to the medical uses, methods of treatment and uses in accordance with the invention.

### BRIEF DESCRIPTION OF THE DRAWINGS

Embodiments of the invention are further described hereinafter with reference to the accompanying drawings, in which:

FIG. 1 shows the proportion and number of mononuclear cells in early and late ARDS compared to healthy controls. (A) shows the proportion and (B) shows the number of circulating monocytes. (C) shows the level of expression of HLA-DR and (D) shows the level of expression of CD11b. Data shown as individual patients with median with interquartile range, significance determined by one-way ANOVA with a Tukey post-test analysis (A, C, D) or Mann-Whitney (B), **P<0.01.

FIG. 2 shows the number of mononuclear cells in LPS induced acute lung injury. (A) shows that circulating blood monocyte numbers are reduced early (24 hours) following LPS-induced ALI in hypoxia (H) compared to normoxia (N) with (B) an associated reduction in lung Ly6C⁺ interstitial macrophages in ALI and (C) in lung Streptococcal infection. Data shown as individual animals with mean±SD, significance determined by one-way ANOVA with a Tukey post-test analysis, **P<0.01, ***P<0.001, ****p<0.0001.

FIG. 3 shows the number of neutrophils and macrophages in LPS-induced ALI. (A) shows that systemic hypoxia (H) is associated with lung neutrophil (Ly6G⁺ cells) persistence, and (B) shows reduced interstitial macrophage numbers (CD64⁺ SiglecF⁻) with complete absence of (C) lung Ly6C⁺ interstitial macrophage populations 5 days following LPS challenge. Data shown as individual animals with mean±SD, significance determined by unpaired t-test (A) one-way ANOVA with a Tukey post-test analysis (B, C), *P<0.05, **P<0.01.

FIG. 4 shows the effects of treatment with CSF1 (0.75 μg/g s.c. daily, days 1-4). (A) shows the proportion and (B) shows the number of circulating monocytes which are both increased in treatment with CSF1. CSF1 also increases the number of (C) lung monocytes, (D) interstitial macrophages and (E) Ly6C⁺ interstitial macrophages. This is associated with a significantly reduced lung neutrophil (F) proportion and (G) numbers of neutrophils, as well as a reduction in (H) bronchoalveolar (BAL) neutrophil numbers, which demonstrates accelerated lung inflammation resolution. Data shown as individual animals with mean±SEM, significance determined by unpaired t-test, *P<0.05, **P<0.01.

FIG. 5 shows the effects of treatment with bone marrow-derived macrophages (BMDM), PBS is the control vehicle: (A) shows a reduction in bronchoalveolar (BAL) neutrophil numbers (B) shows an increase in lung macrophage counts and (C) shows an increase in lung interstitial macrophage counts.

FIG. 6 shows that CSF1 treatment drives a phenotypic switch in blood monocytes and interstitial lung macrophages towards a pro-restorative phenotype and promoting anti-viral type-1 interferon-associated genes: (a) Focused differentially expressed genes (DEG) from classical monocytes isolated from mice treated with LPS and housed in normoxia or hypoxia for 5 days, treated with or without CSF1 were measured by nanostring. (b) Overlap of DEG induced by CSF1 in mice compared to DEG downregulated in ARDS patient monocytes compared to controls.

FIG. 7 shows a Type 1 interferon Kegg pathway analysis of classical monocytes isolated from mice treated with LPS and housed in hypoxia for 5 days, treated with or without CSF1, as measured using the nanostring platform.

FIG. 8 shows an increased proportion of Lyve1+ lung interstitial macrophage (IM) in CSF1-treated hypoxic ALI mice, where Lyve1+ have been shown to be pro-restorative (Chakarov Science 2019; Vol. 363, Issue 6432).

FIG. 9 shows IL-10 measured by MSD V-plex assay as per manufacturer's instructions in serum from LPS-induced ALI mice housed in hypoxia for 5 days and treated with PBS or CSF1 for 4 days.

### DETAILED DESCRIPTION

In one aspect the invention provides an agent for increasing mononuclear cell number in a subject for use in the treatment of a lung disease associated with dysfunctional neutrophilic inflammation.

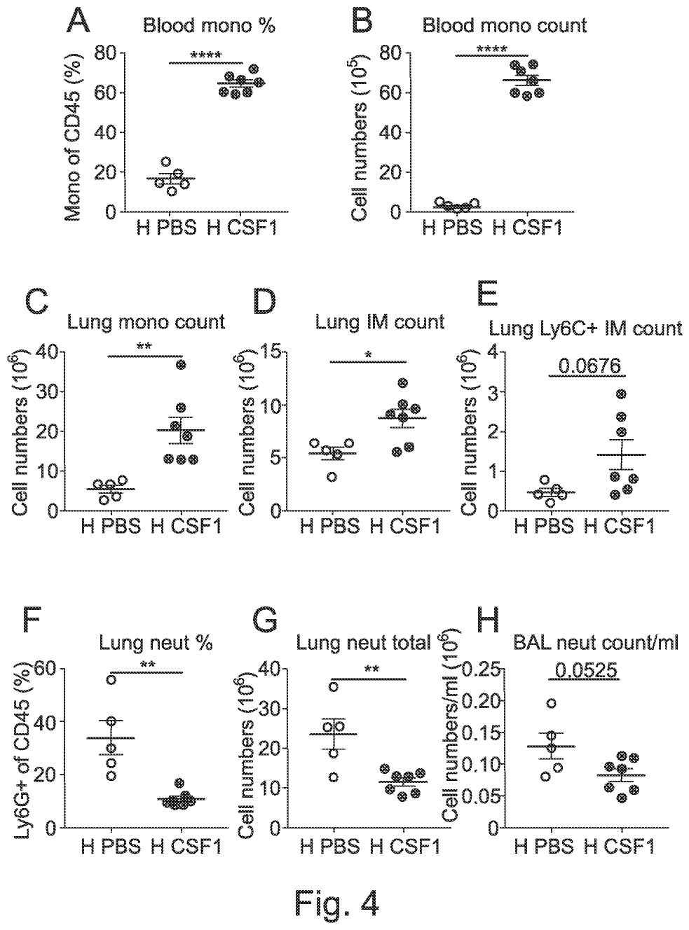

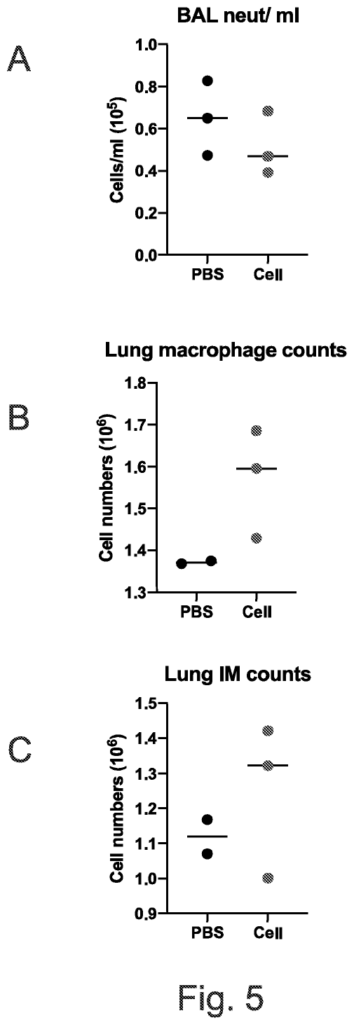

The inventors have surprisingly found that an agent that increases mononuclear cell number can be used to treat a lung disease associated with dysfunctional neutrophilic inflammation (for example ARDS). The inventors have demonstrated that an agent for increasing mononuclear cell number can promote resolution of neutrophilic inflammation in the lung. Without wishing to be bound by theory this may be achieved by clearing neutrophils from the lung. For example, administration of CSF-1 increased monocyte cell number and reduced neutrophil cell number as explained in more detail in the Examples section of the present specification and shown in FIG. 4. Furthermore, direct administration of mononuclear cells such as bone marrow derived macrophages (BMDM) reduced neutrophil cell number and increased lung macrophage cell number, as explained in more detail in the Examples section of the present specification and shown in FIG. 5. The inventors have also surprisingly demonstrated that such agents can increase the number and/or proportion of mononuclear cells in a subject having a pro-restorative phenotype which aids in reducing neutrophil inflammation in the lungs. Therefore, an agent that induces a pro-restorative phenotype in mononuclear cells can be used to treat a lung disease associated with dysfunctional neutrophilic inflammation (for example ARDS). The effectiveness of this approach is surprising, since it was not previously contemplated that such an agent would be useful in the treatment of ARDS and other lung diseases associated with dysfunctional neutrophil inflammation.

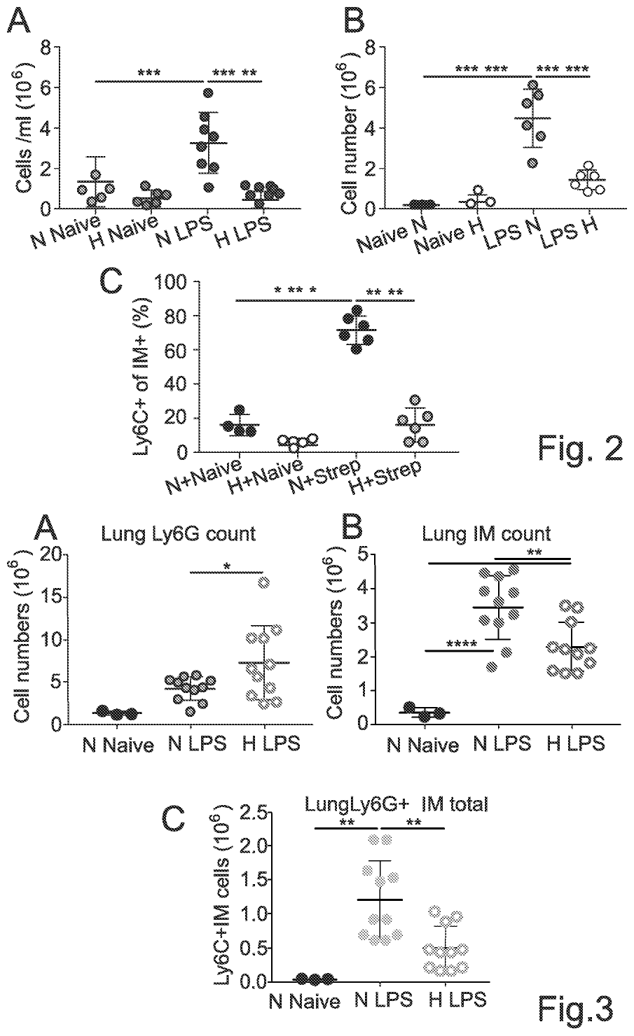

Without wishing to be bound by this hypothesis, the inventors believe that hypoxia, in addition to altering the phenotype of neutrophils, alters the dynamics of mononuclear cells (such as monocytes) leading to a reduced cell count, and alteration in phenotype as shown in the Examples section of the present specification and in FIG. 2. The inventors also believe that this alteration in the dynamics of mononuclear cells may play an important role in the pathogenesis of lung diseases associated with dysfunctional neutrophil inflammation, such as ARDS.

In the context of the present invention, the term “a lung disease associated with dysfunctional neutrophil inflammation” refers to a disease of the lung caused by an increase in number and/or proportion of dysfunctional neutrophils, and/or a decrease in the number and/or proportion of non-dysfunctional neutrophils. Suitably, the dysfunctional neutrophil inflammation may be in the lung.

The term “dysfunctional neutrophil” as used herein refers to a neutrophil with an altered phenotype. Merely by way of example, the altered phenotype may be decreased apoptosis, resistance to phosphoinositide 3-kinase inhibition, increased release of toxic mediators (such as reactive oxygen species, and proteases), increased degranulation, increased NET formation, and/or neutrophil priming. Methods of identifying a dysfunctional neutrophil will be known to those skilled in the art. Merely by way of example, levels of neutrophil apoptosis may be determined by flow cytometry.

Suitably, the lung disease associated with dysfunctional neutrophil inflammation may be ARDS, chronic obstructive pulmonary disease (COPD) or a lung infection. Suitably, the lung infection may be a Streptococcal infection (such as S. pneumoniae infection) or a Staphylococcal infection (such as an S. aureus infection) or a viral infection (such as Influenza A infection or SARS-Cov infection)

In the context of the present specification, the term “agent” refers to a compound which when provided to a subject, results in an increase in mononuclear cell number (for example in the lungs) or an induction of a pro-restorative phenotype in mononuclear cells. The agent may increase the mononuclear cell number directly or indirectly. Agents which are capable of increasing mononuclear cell number or inducing a pro-restorative phenotype in mononuclear cells are well known in the art.

An example of an agent for increasing mononuclear cell number directly may be mononuclear cells themselves. Suitably, the mononuclear cells may be autologous or allogenic. Suitably, the autologous mononuclear cells may be iPSC derived. Suitably, the allogeneic mononuclear cells may be iPSC derived or non-iPSC derived. Allogenic non-iPSC derived mononuclear cells may be obtained, for example, from a healthy blood donor.

Suitably, mononuclear cells may be in a dose from approximately 10⁶ to 10⁹ cells, suitably 10⁷ to 10⁹ cells, suitably at least 10⁶ cells, 10⁷ cells, at least 10⁸, or at least 10⁹ cells. The number of mononuclear cells in each dose may be varied according to the subject to be treated. In some embodiments, each dose comprises at least 1×10⁶ mononuclear cells, at least 1×10⁷ mononuclear cells, suitably at least 1×10⁸, or at least 1×10⁹ mononuclear cells per dose. The subject may be provided with a single dose or multiple doses of mononuclear cells, for example 2, 3, 4, 5 or more doses.

An agent for increasing mononuclear cell number indirectly may modulate a biological process which in turn increases the number of mononuclear cells in the subject. Such a process may increase the production and/or release of mononuclear cells from the bone marrow, and/or expand the lung monocyte/macrophage compartment. Additionally, or alternatively, such a biological process may activate genes (for example CD64, C/EBP-alpha1, MerTK) which are associated with monocyte to macrophage maturation.

Accordingly, an agent for increasing mononuclear cell number indirectly may increase production and/or release of mononuclear cells from the bone marrow, and/or expand the lung monocyte/macrophage compartment. Additionally, or alternatively, the agent may activate genes which are associated with monocyte to macrophage maturation. Genes associated with monocyte to macrophage maturation may be for example selected from the group consisting of CD64, C/EBP-alpha1 and MerTK.

An agent for inducing a pro-restorative phenotype in mononuclear cells may modulate a biological process which in turn induces a phenotypic change in the mononuclear cells. Such a process may increase the number or proportion of pro-restorative mononuclear cells in a subject. This may be achieved by activating certain genes. Suitably by activating certain genes in the type I interferon pathway. Suitably, any genes in the type I interferon pathway may be upregulated in the pro-restorative phenotype. Suitably therefore a pro-restorative mononuclear cell may comprise one or more upregulated genes in the type I interferon pathway. Suitably any of the following genes in the type I interferon pathway may be upregulated in the pro-restorative phenotype: IRF3, IFNAR1, and IFNAR2.

Suitably the pro-restorative mononuclear cells may be pro-restorative monocytes. In such an embodiment, suitably additionally or alternatively, one or more monocyte function genes may be upregulated in the pro-restorative phenotype. Suitably therefore a pro-restorative mononuclear cell may comprise one or more upregulated monocyte function genes. Suitably any monocyte function genes may be upregulated in the pro-restorative phenotype, for example ADGRE1, CCR5 and F480.

Suitably therefore a pro-restorative mononuclear cell may comprise one or more upregulated genes in the type I interferon pathway and one or more upregulated monocyte function genes.

By ‘upregulated’ it is meant that the expression level of a given gene is increased compared to a control mononuclear cell, suitably compared to a mononuclear cell that does not have a pro-restorative phenotype, suitably compared to a mononuclear cell that has not been treated with an agent of the invention. Upregulation of a gene and an increase in gene expression are used interchangeably herein. Gene expression may be measured and compared by any known technique, for example using Nanostring as in the examples contained herein.

Suitably the relevant genes are significantly upregulated compared to a mononuclear cell that does not have a pro-restorative phenotype, suitably compared to a mononuclear cell that has not been treated with an agent of the invention. Suitably the increase in expression of the relevant gene is statistically significant compared to a mononuclear cell that does not have a pro-restorative phenotype, suitably compared to a mononuclear cell that has not been treated with an agent of the invention.

Suitably, expression of the relevant gene is increased by at least 10%, at least 20%, at least 30%, at least 40%, at least 50%, at least 60%, at least 70%, at least 80%, at least 90%, at least 100%, at least 110%, at least 120%, at least 130%, at least 140%, at least 150% compared to a mononuclear cell that does not have a pro-restorative phenotype, suitably compared to a mononuclear cell that has not been treated with an agent of the invention.

Suitably, expression of the relevant gene is increased by at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold compared to a mononuclear cell that does not have a pro-restorative phenotype, suitably compared to a mononuclear cell that has not been treated with an agent of the invention.

In one embodiment, the pro-restorative phenotype comprises upregulated expression of the following genes: IRF3, IFNAR1, and IFNAR2. In such an embodiment, a mononuclear cell having a pro-restorative phenotype comprises upregulated expression of: IRF3, IFNAR1, and IFNAR2.

In one embodiment, the pro-restorative mononuclear cell is a monocyte and the pro-restorative phenotype comprises upregulated expression of the following genes: IRF3, IFNAR1, IFNAR2 and optionally CCR5, ADGRE1 and/or F480. In such an embodiment, a monocyte having a pro-restorative phenotype comprises upregulated expression of: IRF3, IFNAR1, IFNAR2 and optionally CCR5, ADGRE1 and/or F480.

In one embodiment, a mononuclear cell having a pro-restorative phenotype has an increase in expression of IRF3 by at least 2.3 fold.

In one embodiment, a mononuclear cell having a pro-restorative phenotype has an increase in expression of IFNAR1 by at least 1.6 fold.

In one embodiment, a mononuclear cell having a pro-restorative phenotype has an increase in expression of IFNAR2 by at least 1.6 fold.

In one embodiment, a monocyte having a pro-restorative phenotype has an increase in expression of CCR5 of at least 2.5 fold.

In one embodiment, a monocyte having a pro-restorative phenotype has an increase in expression of ADGRE1 of at least 2.5 fold.

Suitably such fold increases are compared to an equivalent mononuclear cell that does not have a pro-restorative phenotype.

Suitably, the induction of a pro-restorative phenotype in monocyte cells leads to an induction in the pro-restorative phenotype in macrophage cells. Suitably a pro-restorative macrophage may be an interstitial pro-restorative macrophage. Suitably a pro-restorative macrophage is Lyve1 positive. In one embodiment, the pro-restorative mononuclear cell may be a lyve1 positive interstitial macrophage. In one embodiment, the pro-restorative mononuclear cell is a macrophage and the pro-restorative phenotype comprises upregulated expression of Lyve1. Suitably the number of pro-restorative Lyve1 positive macrophages is increased by twice, 4 times, 6 times, 8 times, 10 times, 12 times, 14 times, 16 times, 18 times, 20 times, 22 times, 24 times, 28 times, or 30 times compared to the number of pro-restorative Lyve1 positive macrophages in untreated subjects. Suitably the number of pro-restorative Lyve1 positive macrophages in a subject treated with the agent of the invention is between 3-6%, suitably between 3.5-5.5%, suitably about 4%.

Suitably an induction of a pro-restorative phenotype in mononuclear cells may also lead to changes in the environment to be treated. Suitably a pro-restorative phenotype in mononuclear cells may also lead to an increase in environmental IL10. Suitably an increase in serum IL-10 concentration. In some embodiments, the agent for inducing a pro-restorative phenotype in mononuclear cells may also induce an increase in serum IL-10. Suitably the serum IL-10 concentration is increased by twice, 3 times, 4 times, 5 times, 6 times, 7 times, 8 times, 9 times, 10 times, 11 times, 12 times, 13 times, 14 times, or 15 times compared to serum IL-10 concentration in untreated subjects. Suitably the serum IL-10 concentration in a subject treated with the agent of the invention is between 50 to 150 pg/ml, suitably between 75 to 125 pg/ml, suitably between 85 to 115 pg/ml.

The term “mononuclear cell” as used herein refers to a white blood cell with a single nucleus.

Suitably, the mononuclear cell may be selected from the group consisting of a monocyte, a macrophage, and a monocyte precursor. Suitably, the agent for increasing mononuclear cell number increases the number of monocytes and/or macrophages. In one embodiment, “mononuclear cell number” refers to the number of monocytes and/or macrophages.

Suitably, the monocyte may be a circulatory monocyte or a tissue monocyte (for example a lung monocyte), have one or more markers selected from the group consisting of CD45⁺, HLADR⁺, CD14⁺, CD16⁺, CD11b⁺, CD206⁻, CD169⁻, and/or be non-granulocytic.

Suitably, the macrophage may be a monocyte derived macrophage and/or a tissue macrophage. Suitably the monocyte derived macrophage and/or tissue macrophage may be a lung macrophage. Suitably the monocyte derived macrophage may be a bone marrow-derived macrophage, or a peripheral blood monocyte (PBMC) derived macrophage.

Suitably, the monocyte derived macrophage may have one or more markers associated with or characteristic of an alveolar macrophage. Suitably, the monocyte derived macrophage may have one or more markers selected from the group consisting of CD11b⁺, HLA-DR⁺, CD206⁺, and CD169⁺. Optionally, the alveolar macrophage may be CD15⁻.

Suitably, the monocyte derived macrophage may have one or more markers associated with or characteristic of an interstitial macrophage. Suitably, the monocyte derived macrophage may have one or more markers selected from the group consisting of CD11b⁺, HLA-DR⁺, CD206⁺, CD169⁻. Optionally, the interstitial macrophage may be CD15⁻.

The presence, level or absence of a marker polypeptide or nucleic acid molecule (e.g. mRNA) in a population of macrophages can be determined by contacting the sample population with a compound or an agent capable of specifically detecting (e.g. specifically binding) the specific marker polypeptide or nucleic acid molecule.

Routine methods may be used to obtain sample from a cell population. For example, by immersing the cell population in a buffer for extracting protein or mRNA.

The level of any specific marker in a cell population can be measured in a number of ways, including: measuring the mRNA that encodes the protein marker; measuring the amount of protein marker; or measuring the activity of the protein biomarker.

Any known mRNA detection method may be used to detect the level of mRNA of a marker of interest (e.g. CD11b, HLA-DR, CD206, CD169) in a sample.

For example, the level of a specific mRNA in a sample can be determined both by in situ and by in vitro formats. mRNA may be detected using Northern blot analysis, polymerase chain reaction, probe arrays or RNA sequencing. In one embodiment, a sample may be contacted with a nucleic acid molecule (i.e. a probe, such as a labeled probe) that can specifically hybridize to the specific mRNA of the marker of interest (e.g. CD11b, HLA-DR, CD206, CD169). The probe may be, for example, a complement to a full-length nucleic acid molecule, or a portion thereof, such as a nucleic acid molecule of at least 10, 15, 30, 50, 100, 250 or 350 nucleotides in length and which specifically hybridizes under stringent conditions to specific mRNA of interest.

The term “hybridisation” as used herein shall include “the process by which a strand of nucleic acid joins with a complementary strand through base pairing” as well as the process of amplification as carried out in polymerase chain reaction (PCR) technologies. Hybridisation conditions are based on the melting temperature (Tm) of the nucleotide binding complex, as taught in Berger and Immel (1987, Guide to Molecular Cloning Techniques, Methods in Enzymology, Vol. 152, Academic Press, San Diego Calif.), and confer a defined “stringency” as explained below. Maximum stringency typically occurs at about Tm−5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridisation can be used to identify or detect identical nucleotide sequences while an intermediate (or low) stringency hybridisation can be used to identify or detect similar or related polynucleotide sequences. In a preferred aspect, the present invention covers the use of nucleotide sequences that can hybridise to the nucleotide sequences discussed herein, or the complement thereof, under stringent conditions (e.g. 50° C. and 0.2×SSC). In a more preferred aspect, the present invention covers the use of nucleotide sequences that can hybridise to the nucleotide sequences discussed herein, or the complement thereof, under high stringency conditions (e.g. 65° C. and 0.1×SSC).

Alternatively, the level of a specific mRNA in a sample may be evaluated with nucleic acid amplification, for example by RT-PCR, ligase chain reaction, self-sustained sequence replication, transcriptional amplification or any other nucleic acid amplification method, followed by the detection of the amplified molecules using techniques known in the art including RNA sequencing.

Suitably, the level of at least one of one or more markers (e.g. CD11b, HLA-DR, CD206, CD169) may be measured by RT-PCR analysis.

Any known protein detection method may be used to detect the level of protein of a marker of interest (e.g. CD11b, HLA-DR, CD206, CD169) in a sample.

Generally, protein detection methods comprise contacting an agent that selectively binds to a protein, for example an anti-CD11b an anti-CD169, an anti-HLA-DR or an anti-CD206, with a sample to determine the level of the specific protein in the sample. Preferably, the agent or antibody is labeled, for example with a detectable label. Suitable antibodies may be polyclonal or monoclonal. An antibody fragment such as a Fab or F(ab′)2 may be used.

As used herein the term “labeled”, refers to direct labeling of the probe or antibody by coupling (i.e., physically linking) a detectable substance to the probe or antibody, as well as indirect labeling of the probe or antibody by reactivity with a detectable substance.

The level of a specific protein marker in a sample may be determined by techniques known in the art, such as enzyme linked immunosorbent assays (ELISAs), immunoprecipitation, immunofluorescence, enzyme immunoassay (EIA), radioimmunoassay (RIA), Western blot analysis, Flow cytometry and Lateral Flow Devices (LFDs) utilizing a membrane bound antibody specific to the protein biomarker. Alternatively, the level of a specific biomarker protein in a sample can be detected and quantified using mass spectrometry. Such methods are routine in the art.

Accordingly, it will be appreciated, that in the context of the present invention, the agent is for increasing the cell number of a mononuclear cell having any one or more of the aforementioned characteristics. It will also be appreciated that the agent itself may be a mononuclear cell having any one or more of the aforementioned characteristics.

Suitably, in aspects where the number of monocytes or monocyte precursors are increased, the monocytes or the monocyte precursors may mature into monocyte derived macrophages. The maturation may occur in vivo. Thus, it will be appreciated that whilst the agent may initially increase the number of monocytes or monocyte precursors (for example if the agent is a monocyte or a monocyte precursor), it may lead to an increase in the number of monocyte derived macrophages. Thus, whilst the agent may initially increase the number or proportion of pro-restorative monocytes, it may lead to an increase in the number or proportion of pro-restorative macrophages. Suitably, the number of monocyte derived macrophages may be increased 1 day, 2 days, 3 days, 4 days, 5 days, 6 days, 7 days, 8 days, 9 days, 10 days, 11 days, 12 days, 13 days, 14 days or more, after the provision of monocytes or monocyte precursors.

Various methods of producing macrophages from pluripotent stem cells such as ES cells and iPSCs are known in the art, see Yeung et al. 2012 (“Conditional-ready mouse embryonic stem cell derived macrophages enable the study of essential genes in macrophage function”. Sc. Rep. 2015 Mar. 10; 5:8909. doi: 10.1038/srep08908) or Sneju et al. (“Application of iPS cell-derived macrophages to cancer therapy” Oncoimmunology. 2014; 3: e27927). Methods of producing monocyte derived macrophages are also known in the art, see van Wgenburt et al., (“Efficient, Long Term Production of Monocyte-Derived Macrophages from Human Pluripotent Stem Cells under Partly-Defined and Fully-Defined Conditions” PLOS ONE 8(8): e71098. doi:10.1371/journal.pone.0071098). Macrophages produced using such methods may be used as agents for increasing mononuclear cells in the context of the present invention.

ESC derived macrophages (ESDMs) may be generated by culturing the ESCs in the presence of colony stimulating factor-1 (CSF-1) (also known as M-CSF) and IL-3 to form embryoid bodies (EB). Whilst EBs adhere to tissue culture plastic, macrophage progenitor cells are non-adherent and thus are released into the medium. The macrophage progenitor cells may then be harvested at various time points, for example after 10 or 20 days and plated onto non-treated Petri dishes and cultured in the presence of CSF-1 alone. This process can give rise to monocyte-like cells that adhere to the plastic forming a monolayer and mature into ESDM. The maturation of the ESC into ESDM can be monitored by detecting the presence of mature macrophage specific markers F4/80 (mouse macrophage specific) or 25F9 (human macrophage specific) and CD11b. Advantageously, the method described yields a substantially homogenous population of ESDMs. Suitably, ESDMs for use in the invention may be human ESCs and hence the marker 25F9, optionally in combination with CD11b can be used to determine maturation into ESDMs. Optional further markers may be selected from one or more of the group consisting of: CD45⁺, HLADR⁺, CD14⁺, CD16⁺, CD11b⁺ and CD206⁺.

PBMC derived macrophages may be generated by culturing the PBMCs in the presence of colony stimulating factor-1 (CSF-1) (also known as M-CSF) to form differentiated macrophages. The PBMCs may be harvested and cultured in TexMACS medium (Miltenyi) supplemented with 100 ng/mL M-CSF (R&D Systems) for 7 days, under a humidified atmosphere at 37° C., with 5% CO₂. A 50% volume media replenishment may be carried out twice during culture (days 2 and 4) with 50% of the culture medium removed, then fed with fresh medium supplemented with 200 ng/mL M-CSF (to restore a final concentration of 100 ng/mL). The maturation of PBMCs into macrophages can be monitored by detecting specific markers such as CD45 and CD14 for lineage determination and 25F9 as a marker of macrophage maturity. In addition CD206 as a marker for phagocytosis and scavenging capacity, and further markers CD163, and CD169.

Alternatively, macrophages may be derived from iPSC. Suitably, the method for differentiation of iPSCs to macrophages may involve supplementing culture medium with a cytokine Mix 1 (comprising bone morphogenetic protein (BMP4), vascular endothelial growth factor (VEGF) and stem cell factor (SCF)). Cells may be cut, dislodged, divided and re-cultured in fresh media supplemented with cytokine mix 1. Cells may be cultured in suspension for 3 days with a cytokine top up on Day 2, to form EBs. The EBs may then be transferred to media supplemented with cytokine Mix 2 (comprising M-CSF, IL3, Glutamax, Penicillin/Streptomycin and β-mercaptoethanol). EBs can be maintained in this medium for the remaining duration of the protocol, with spent medium being replaced with fresh medium every 3-4 days. After about 2 weeks, the EBs produced macrophage progenitors in the culture supernatant that were harvested and transferred to medium supplemented with cytokine Mix 3 (M-CSF, Glutamax, Penicillin/Streptomycin) and allowed to mature into iPSC-derived macrophages (iPSC-DM). Macrophage progenitors may continue to be harvested twice a week for approximately 2 months.

Suitably, an agent for indirectly increasing the mononuclear cell count or inducing a pro-restorative phenotype in the mononuclear cells may be selected from the group consisting of a polypeptide, a nucleic acid, a mononuclear cell, a drug, or any combination thereof.

An agent for increasing the mononuclear cell number indirectly or for inducing a pro-restorative phenotype in the mononuclear cells, may be selected from the group consisting of a cytokine, an enzyme, a hormone, a growth factor, a regulatory protein, and an immunoregulator, or a fragment, variant or homolog thereof.

Suitably, the cytokine may be selected from the group consisting of CSF-1 and IL-34. More suitably, the cytokine is CSF1.

As used herein, the term “CSF1” refers to Colony Stimulating Factor 1. CSF1 is a cytokine that plays a role in the regulation of survival, proliferation and differentiation of haematopoietic precursor cells, especially mononuclear cells, such as macrophages and monocytes. In the present specification, the terms “CSF-1”, “M-CSF”, “macrophage colony stimulating factor”, “CSF1”, “colony stimulating factor1” and “colony stimulating factor-1” are used interchangeably herein. Suitably CSF1 may human or porcine.

In a suitable embodiment, the polypeptide may comprise or consist of an amino acid sequence that shares at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 96% identity, at least 98% identity, at least 99% identity with SEQ ID NO: 1 or a fragment, variant or homolog thereof. Suitably, CSF1 may be a polypeptide comprising or consisting of an amino acid sequence according to SEQ ID NO:1 or a fragment, variant or homolog thereof.

The term “fragment” as used herein refers to a polypeptide that consists of a truncation of the corresponding wild type amino acid. A fragment of the polypeptide may share 100% identity with the portion of the wild type amino acid sequence that it corresponds to.

A suitable fragment of CSF1 may consists up to 549 contiguous amino acids of SEQ ID NO: 1, for example up to 500 contiguous amino acids of SEQ ID NO: 1, up to 450 contiguous amino acids of SEQ ID NO: 1, up to 400 contiguous amino acids of SEQ ID NO: 1, up to 350 contiguous amino acids of SEQ ID NO: 1, up to 300 contiguous amino acids of SEQ ID NO: 1, up to 250 contiguous amino acids of SEQ ID NO: 1, up to 200 contiguous amino acids of SEQ ID NO: 1, up to 150 contiguous amino acids of SEQ ID NO: 1, up to 100 contiguous amino acids of SEQ ID NO: 1, up to 50 contiguous amino acids of SEQ ID NO: 1, or fewer than 50 contiguous amino acids of SEQ ID NO: 1. More suitably, the fragment may consist of between 100 and 200 contiguous amino acids of SEQ ID NO: 1. More suitably the fragment may consist of approximately 150 contiguous amino acids of SEQ ID NO: 1, for example 154 contiguous amino acids of SEQ ID NO: 1. In a suitable embodiment, the CSF1 fragment may be as defined in WO2014/132072. In a suitable embodiment, the CSF1 fragment may be as defined in Gow et al. In a suitable embodiment, the CSF1 fragment may consist of amino acid residues 36 to 190 of SEQ ID NO:1. In a suitable embodiment, the CSF1 fragment may consist of amino acid residues 33 to 182 of SEQ ID NO:1.

In a suitable embodiment, the CSF1 fragment may be a part of a fusion protein. The fusion protein may further comprise biologically active antibody fragment. Accordingly, in such an embodiment, the agent for increasing the mononuclear cell number is a fusion protein.

“Fusion protein” as used herein, refers to a protein produced when two heterologous nucleotide sequences or fragments thereof coding for two (or more) different polypeptides, or fragments thereof, are fused together in the correct translational reading frame. The two or more different polypeptides, or fragments thereof, include those not found fused together in nature and/or include naturally occurring mutants.

Suitably the antibody is an immunoglobulin selected from the group comprising IgA, IgD, IgE, IgG and IgM more preferably it is IgG. Suitably, the antibody fragment may be a fragment of the porcine IgG1a.

Suitably, the antibody fragment is selected from the group comprising F(ab′)2, Fab′, Fab, Fv, Fc and rIgG and more preferably it is an Fc fragment.

Suitably, the fragment of CSF-1 or variant or homolog thereof and the biologically active antibody fragment of the fusion protein are covalently linked directly or through a linker moiety.

In a suitable embodiment, the fusion protein may comprise or consist of a sequence that shares at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 96% identity, at least 98% identity, at least 99% identity with SEQ ID NO: 4. Suitably, the fusion protein comprises the amino acid sequence of SEQ ID NO:4. Suitably, the fusion protein may comprise or consist of the amino acid sequence of SEQ ID NO:4.

In a suitable embodiment, the fusion protein may be as defined in WO2014/132072. In a suitable embodiment, the fusion protein may be as defined in Gow et al.

Suitably, the CSF1 fragment is according to SEQ ID NO: 2.

Suitably the antibody fragment is according to SEQ ID NO: 3.

As used herein, the term “variant” refers to a polypeptide in which one or more amino acid have been replaced by different amino acids as compared to the corresponding wild type amino acid sequence. It is well understood in the art that some amino acids may be changed to others with broadly similar properties without changing the nature of the activity of the polypeptide (conservative substitutions). Generally, the substitutions which are likely to produce the greatest changes in a polypeptide's properties are those in which (a) a hydrophilic residue (e.g., Ser or Thr) is substituted for, or by, a hydrophobic residue (e.g. Leu, lie, Phe or Val); (b) a cysteine or proline is substituted for, or by, any other residue; (c) a residue having an electropositive side chain (e.g., Arg, His or Lys) is substituted for, or by, an electronegative residue (e.g., Glu or Asp) or (d) a residue having a bulky side chain (e.g., Phe or Trp) is substituted for, or by, one having a smaller side chain (e.g., Ala, Ser) or no side chain (e.g., Gly).

As used herein, the term “homolog” refers to a polypeptide that shares a definable amino acid sequence relationship with the corresponding wild type amino acid sequence.

It will be appreciated that in the context of the present invention, the fragment, variant or homolog substantially retains the biological activity of the corresponding wild type polypeptide. The term “biological activity” as used herein refers to the ability to increase mononuclear cell number in a subject. By “substantially retains” biological activity, it is meant that the fragment retains at least about 50%, 60%, 75%, 85%, 90%, 95%, 97%, 98%, 99%, or more, of the biological activity of the wild type polypeptide to increase mononuclear cell number. Indeed, the fragment, variant or homolog may have a higher biological activity than the wild type polypeptide. Suitably, the fragment, variant or homolog may have 110%, 120%, 130%, 140%, 150%, 160%, 170%, 180%, 190%, 200%, or more, of the biological activity of the wild type polypeptide to increase mononuclear cell number.

The term “nucleic acid” as used herein relates to any DNA, RNA or other nucleotide, or any analogue or derivative thereof. Suitably, the RNA may be a miRNA. In a suitable embodiment the nucleic acid may be an expression vector comprising a sequence encoding a polypeptide, or a fragment, variant or homolog thereof. Suitably, the expression vector may comprise a sequence encoding CSF1, or a fragment, variant or homolog thereof as defined elsewhere in this specification.

The term “small molecule” as used herein refers to a synthetic drug molecule.

The term “increasing mononuclear cell number” refers to increasing the proportion and/or total number of mononuclear cells in the subject. Methods for determining proportion and/or total number of mononuclear cells in the subject will be known to those skilled in the art. Merely by way of example, such methods may involve a white blood cell count, or white blood cell flow cytometric analysis. It will be appreciated that mononuclear cell number may be considered sufficiently increased when the proportion and/or total number of mononuclear cells in the subject is within a reference range. Merely by way of example, the reference range may be based on the proportion and/or total number of mononuclear cells in a healthy control. Suitably, the proportion and/or total number of mononuclear cells in the subject may be measured by flow cytometry. Advantageously, the use of flow cytometry allows the distinction between immature neutrophils and monocytes.

In the context of the present invention, the term ‘pro-restorative phenotype’ as used herein refers to a mononuclear cell with a phenotype which promotes repair, such a phenotype suitably includes the increased expression of certain genes which are defined hereinabove. Suitably a pro-restorative phenotype may comprise an increase in expression of any gene in the type I interferon pathway. Suitably a pro-restorative phenotype may comprise an increase in expression of any of the following genes: IRF3, IFNAR1, and IFNAR2. Suitably a pro-restorative phenotype in a monocyte may comprise an increase in expression of any of the following genes: IRF3, IFNAR1, and IFNAR2 and optionally any gene involved in monocyte function, such as CCR5, ADGRE1, and/or F480. Suitably a pro-restorative phenotype in a macrophage may comprise an increase in expression of Lyve1. ‘Inducing a pro-restorative phenotype’ in mononuclear cells refers to increasing the proportion and/or total number of mononuclear cells having a pro-restorative phenotype in the subject. Methods for determining those mononuclear cells with a pro-restorative phenotype are known to those skilled in the art. Such methods may include using the Nanostring platform for analysing gene expression as explained in the examples.

As used herein the term “subject” refers to an individual diagnosed with or likely to have a lung disease associated with dysfunctional neutrophil inflammation (for example ARDS or COPD). Merely by way of example, a subject may be identified as being likely to have a lung disease associated with dysfunctional neutrophil inflammation (for example ARDS or COPD) if the subject has septicaemia, pneumonia, severe flu, or experienced near drowning, and/or if found to have a reduced mononuclear cell count.

Suitably, the subject may be a mammal. Suitably, the subject may be human.

The agent for increasing mononuclear cell number or for inducing a pro-restorative phenotype in mononuclear cells may be provided to the subject as a first line treatment for a lung disease associated with dysfunctional neutrophil inflammation. In such an embodiment, the subject would have not been provided any other treatment for a lung disease associated with dysfunctional neutrophil inflammation, prior to treatment in accordance with the present invention. Suitably, the agent for increasing mononuclear cell number or for inducing a pro-restorative phenotype in mononuclear cells as a first line treatment may be provided as a first line of treatment in conjunction with a further treatment. Such a further treatment may be, for example, oxygen therapy. It will be appreciated that the use of an agent for increasing mononuclear cell number or for inducing a pro-restorative phenotype in mononuclear cells in conjunction with the further treatment may have a synergistic effect.

Alternatively, the agent for increasing mononuclear cell number or for inducing a pro-restorative phenotype in mononuclear cells may be provided as a second line of treatment. Merely by way of example, in such an embodiment, lung protective ventilatory strategies and/or fluid-conservation strategies may be the first line of treatment.

It will be appreciated that when the lung disease associated with dysfunctional neutrophil inflammation (such ARDS) is accompanied or caused by an underlying condition, such as an infection (for example pneumonia, septicaemia, or severe flu), the agent may be provided alongside a treatment for the underlying conditions, for example antibiotic or antiviral treatment.

The term “providing” as used herein encompasses any technique by which the subject receives a therapeutically effective amount of the agent for increasing mononuclear cell number.

It will be appreciated that there are various routes in which the subject may be provided with a therapeutically effective amount of the agent for increasing mononuclear cell number. Such suitable routes may be selected from the group consisting of: intravenous, intratracheal, parenteral, subcutaneous, intraperitoneal, intramuscular, intravascular, intranasal, rectal, transdermal, percutaneous and oral.

Suitably, the agent or pharmaceutical composition comprising the agent may be formulated for a delivery selected from the group consisting of: intravenous, intratracheal, parenteral, subcutaneous, intraperitoneal, intramuscular, intravascular, intranasal, rectal, transdermal, percutaneous and oral.

The term “therapeutically effective amount” as used herein, refers to the amount of the agent for increasing mononuclear cell number or for inducing a pro-restorative phenotype in mononuclear cells, that when provided to the subject, is sufficient to treat a lung disease associated with dysfunctional neutrophil inflammation. The terms “treat”, “treating” or “treatment” refer to a clinical improvement of a lung disease associated with dysfunctional neutrophil inflammation in a subject with this disease. Such a clinical improvement may be demonstrated by an improvement of the pathology and/or symptoms associated with the disease. Suitably, clinical improvement may be demonstrated by partial or complete reversal of the disease.

Clinical improvement of the pathology and/or symptoms may be demonstrated for example by one or more of the following: reduction of neutrophils (e.g. dysfunctional neutrophils) in the lung, reduction of oedema in the lung, reduction of hypoxia and/or improved respiratory rate. Other signs of clinical improvement will be known to the skilled person. It will be appreciated that the therapeutically effective amount will vary depending on various factors, such as the subject's body weight, sex, diet and route by which the agent is provided. It will be appreciated that in the context of the present invention, reduction of neutrophils may be due to increased clearance of neutrophils from the lung as opposed to mere prevention or reduction of neutrophil infiltration.

A therapeutically effective amount may be provided to the subject in a single dose or multiple doses.

In the context of the present invention the agent may be in the form of a pharmaceutical composition. In a suitable embodiment, a pharmaceutical composition may comprise in addition to the agent for increasing mononuclear cell number or for inducing a pro-restorative phenotype in mononuclear cells, a pharmaceutically acceptable concentration of salt, buffering agents, and compatible carriers. The compositions may also include antioxidants and/or preservatives. Suitable antioxidants may be selected from the group consisting of: mentioned thiol derivatives (e.g. thioglycerol, cysteine, acetylcysteine, cystine, dithioerythreitol, dithiothreitol, glutathione), tocopherols, butylated hydroxyanisole, butylated hydroxytoluene, sulfurous acid salts (e.g. sodium sulfate, sodium bisulfite, acetone sodium bisulfite, sodium metabisulfite, sodium sulfite, sodium formaldehyde sulfoxylate, sodium thiosulfate) and nordihydroguaiareticacid. Suitable preservatives may for instance be phenol, chlorobutanol, benzylalcohol, methyl paraben, propyl paraben, benzalkonium chloride and cetylpyridinium chloride.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise. Accordingly, it will be appreciated that in the context of the present specification, where reference is made to “a mononuclear cell” this includes a population of mononuclear cells.

In a further aspect, the invention provides Colony Stimulating Factor 1 (CSF1), or a fragment, variant or homolog thereof, for use in the treatment of a lung disease associated with dysfunctional neutrophilic inflammation.

Suitably, the CSF1 may comprise or consist of an amino acid sequence that shares at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 96% identity, at least 98% identity, at least 99% identity with SEQ ID NO: 1 or a fragment, variant or homolog thereof. Suitably, CSF1 may be a polypeptide comprising or consisting of an amino acid sequence according to SEQ ID NO:1 or a fragment, variant or homolog thereof.

Suitably CSF1 may human or porcine.

The term “fragment” as used herein refers to a polypeptide that consists of a truncation of the corresponding wild type amino acid. A fragment of the polypeptide may share 100% identity with the portion of the wild type amino acid sequence that it corresponds to.

A suitable fragment of CSF1 may consists up to 549 contiguous amino acids of SEQ ID NO: 1, for example up to 500 contiguous amino acids of SEQ ID NO: 1, up to 450 contiguous amino acids of SEQ ID NO: 1, up to 400 contiguous amino acids of SEQ ID NO: 1, up to 350 contiguous amino acids of SEQ ID NO: 1, up to 300 contiguous amino acids of SEQ ID NO: 1, up to 250 contiguous amino acids of SEQ ID NO: 1, up to 200 contiguous amino acids of SEQ ID NO: 1, up to 150 contiguous amino acids of SEQ ID NO: 1, up to 100 contiguous amino acids of SEQ ID NO: 1, up to 50 contiguous amino acids of SEQ ID NO: 1, or fewer than 50 contiguous amino acids of SEQ ID NO: 1. More suitably, the fragment may consist of between 100 and 200 contiguous amino acids of SEQ ID NO: 1. More suitably the fragment may consist of approximately 150 contiguous amino acids of SEQ ID NO: 1, for example 154 contiguous amino acids of SEQ ID NO: 1. In a suitable embodiment, the CSF1 fragment may be as defined in WO2014/132072. In a suitable embodiment, the CSF1 fragment may be as defined in Gow et al. In a suitable embodiment, the CSF1 fragment may consist of amino acid residues 36 to 190 of SEQ ID NO:1. In a suitable embodiment, the CSF1 fragment may consist of amino acid residues 33 to 182 of SEQ ID NO:1.

In the context of some aspects of the present invention, CSF1 or a fragment, variant or homolog thereof may be a part of a fusion protein. The fusion protein may further comprise biologically active antibody fragment.

“Fusion protein” as used herein, refers to a protein produced when two heterologous nucleotide sequences or fragments thereof coding for two (or more) different polypeptides, or fragments thereof, are fused together in the correct translational reading frame. The two or more different polypeptides, or fragments thereof, include those not found fused together in nature and/or include naturally occurring mutants.

Suitably the antibody is an immunoglobulin selected from the group comprising IgA, IgD, IgE, IgG and IgM more preferably it is IgG. Suitably, the antibody fragment may be a fragment of the porcine IgG1a.

Suitably, the antibody fragment is selected from the group comprising F(ab′)2, Fab′, Fab, Fv, Fc and rIgG and more preferably it is an Fc fragment.

Suitably, CSF-1 or fragment, variant or homolog thereof and the biologically active antibody fragment of the fusion protein are covalently linked directly or through a linker moiety.

In a suitable embodiment, the fusion protein may be as defined in WO2014/132072. In a suitable embodiment, the fusion protein may be as defined in Gow et al.

In a suitable embodiment, the fusion protein may comprise or consist of a sequence that shares at least 75% identity, at least 76% identity, at least 77% identity, at least 78% identity, at least 79% identity, at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 96% identity, at least 98% identity, at least 99% identity with SEQ ID NO: 4. Suitably, the fusion protein comprises the amino acid sequence of SEQ ID NO:4. Suitably, the fusion protein consists of the amino acid sequence of SEQ ID NO:4.

Suitably, the CSF1 fragment is according to SEQ ID NO: 2.

Suitably, the antibody fragment is according to SEQ ID NO: 3.

Suitably, Colony Stimulating Factor 1 (CSF1), or a fragment, variant or homolog thereof may increase monocyte and/or macrophage cell number in the lungs of the subject.

Suitably, the lung disease associated with dysfunctional neutrophilic inflammation may be selected from the group consisting of acute respiratory distress syndrome (ARDS), chronic obstructive pulmonary disease (COPD) and lung infection.

Suitably, the dysfunctional neutrophilic inflammation may be in the lung.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

In a further aspect, the invention provides Colony Stimulating Factor 1 (CSF1), or a fragment, variant or homolog thereof, for use in the treatment of a lung disease associated with dysfunctional neutrophilic inflammation by increasing the mononuclear cell number in a subject.

In a further aspect, the invention provides Colony Stimulating Factor 1 (CSF1), or a fragment, variant or homolog thereof, for use in the treatment of a lung disease associated with dysfunctional neutrophilic inflammation by inducing a pro-restorative phenotype in mononuclear cells of the subject.

Various further aspects and embodiments of the present invention will be apparent to those skilled in the art in view of the present disclosure.

All documents mentioned in this specification are incorporated herein by reference in their entirety.

### EXAMPLES

### Example 1

### 1 Material and Methods

### Human Healthy Control Blood Donors

ARDS patients were recruited and informed consent obtained by proxy under the METACYTE study (17/SS/0136/AM01). AM healthy participants gave written informed consent in accordance with the Declaration of Helsinki principles, with AMREC approval for the study of healthy human volunteers through the MRC/University of Edinburgh Centre for Inflammation Research blood resource (15-HV-013). Up to 40 mls of whole blood was collected into citrate tubes and up to 10 million cells were stained for flow cytometry assessment and sorting. Briefly, the whole blood was treated with red cell lysis buffer (Invitrogen) and cells counted prior to staining for flow cytometry. Cells were incubated with anti-CD16/32 Fc-block (1:50) for 30 minutes followed by staining for 30 minutes with antibodies (see Table 1) followed by a wash with FACS buffer. Dapi (1:1000) was added prior to flow cytometry.

### Animals

Male C57/BL6 mice aged 6-8 weeks were purchased from Harlan for use in the LPS-induced lung inflammation model. Animal experiments were conducted in accordance with the UK Home Office Animals (Scientific Procedures) Act of 1986 with local ethical approval.

### Mouse LPS Acute Lung Injury Model

Nebulized LPS (3 mg) was administered to awake mice, which were then housed in normoxia or hypoxia (10% 02) for up to 5 days. Mice were treated with Colony Stimulating Factor (CSF)-1-Fc by subcutaneous injection (0.75 μg/g/mouse) from day 1 to 4 post-LPS, prior to cull on day 5. Mice were culled with an overdose of intraperitoneal anaesthetic (Euthetal) followed by blood collection from the inferior vena cava. Alveolar leukocytes were collected by bronchoalveolar lavage (BAL), then mice were perfused lightly with PBS through the heart, prior to harvest of lung tissue. On occasion, one femur was harvested for bone marrow leukocyte assessment.

Tissue leukocytes were extracted from surgically dissociated king tissue by enzymatic digestion with 2 ml enzyme mix (RPMI with 0.625 mg/ml collagenase D (Roche), 0.85 mg/ml collagenase V (Sigma-Aldrich), 1 mg/ml dispase (Gibco, Invitrogen) and 30 U/ml DNase (Roche Diagnostics GmbH), collagenase V (Sigma), collagenase D (Roche), dispase (Gibco) and DNase (Roche)) for 45 minutes at 37° C. in a shaking incubator. The digest material was passed through a 100 μM cell strainer with the addition of FACS buffer (PBS with 0.5% BSA and 0.02 mM EDTA). Cell pellets were treated with red cell lysis buffer (Sigma) and washed in FACS buffer. The resulting cell suspension was subsequently passed through a 40 μm strainer before cell counting using a Casey TT counter (Roche). Single cell suspensions (1 million cells/sample) were then stained for flow cytometry. BAL samples were counted prior to staining for flow cytometry. Mouse blood and bone marrow were treated with red blood cell lysis buffer (Biolegend) prior to counting and staining for flow cytometry (see Table 1).

### Mouse Pneumonia Model

Animals were anaesthetised using a ketamine/medetomidine mix injected intraperitoneally. The mice were then given S. pneumoniae (mD39 strain) 1×10⁷ colony forming units (c.f.u.) in 50 μl via the intratracheal cannula. Following anaesthetic reversal (Antisedan), mice were monitored closely for 3 hours until awake, after which they were placed in either normoxia (21% FiO2) or hypoxia (10%) for 21 hours. Blood and lungs were collected for flow cytometry.

### Flow Cytometry

Mouse cells were treated with α-CD16/32 Fc block (e-bioscience) prior to staining with antibodies (see supplementary table 1). Relevant full minus one (FMO) samples were used as controls. Zombie Aqua fixable viability dye (Biolegend) was used to exclude dead cells from tissue samples or Dapi for single cell suspensions. Cells were acquired on the LSRFortessa or Calibur (Becton Dickinson). Compensation was performed using BD FACSDiva software and data analysed in FlowJo version 10.

### Cytokine/Chemokine Quantification

BAL and serum supernatants were collected and stored at −80 until use. Cytokine and chemokine levels were measured using an MSD V-plex plate as per manufacturer's instructions.

### Nanostring Platform Gene Quantification

5000 mouse classical monocytes were sorted from mice treated with LPS and housed in normoxia, hypoxia and hypoxia+CSF1 gating on Single Dapi⁻CD45⁺Lin⁻CD115⁺Ly6C^{bright} cells into RLT. Cell pellets were frozen until ready for processing. Mouse myeloid inflammation NanoString gene expression plates were run as per manufacturer's instructions at the University of Edinburgh HTPU Centre within MRC Institute of Genetics and Molecular Medicine/Cancer Research UK Edinburgh Centre.

### Gene Expression Analysis

Normalisation of data was carried out using the geNorm selection of housekeeping genes function on NanoString nCounter analysis software. Resulting Log 2 normalised values were used in subsequent analyses. Differential genes (“DE genes) were defined as genes with log 2 FC>1, P-value<0.05 across sample groups. Hierarchical clustering of sets of DE genes was carried out using Euclidian and Ward methods based on Pearson correlation values across transcriptional scores. Z-score scalar normalisation of data was applied to the data prior to plotting as heatmaps.

### Quantification and Statistical Analysis

Statistical tests were performed using Prism 7.00 software (GraphPad Software Inc) (specific tests detailed in figure legends). Significance was defined as a p value of <0.05 (after correction for multiple comparisons where applicable). Significance values are summarized as follows: *p<0.05, **p<0.01, ***p<0.001. Sample sizes (with each n number representing a different blood donor for human cells or an individual mouse for animal experiments) are shown in figure legends.

TABLE 1
Antibodies used in flow cytometry analysis

| Antibody | Clone | Source | Dilution | |
|---|---|---|---|---|
| CD16 | eBioCD16 | eBioscience | 5 | ul/sample |
| CD3 | OKT3 | Biolegend | 1.25 | ul/sample |
| CD56 | HCD56 | Biolegend | 1.25 | ul/sample |
| CD19 | HIB19 | Biolegend | 2.5 | ul/sample |
| CCR2 | KO36C2 | Biolegend | 5 | ul/sample |
| ICAM | HCD54 | Biolegend | 5 | uL/sample |
| CD45 | 2DI | Biolegend | 5 | ul/sample |
| CD14 | M5E2 | Biolegend | 5 | ul/sample |
| HLA-DR | L243 | Biolegend | 5 | uL/sample |

| | | | |
|---|---|---|---|
| SiglecF | E50-2440 | BD | 1:800 |
| CD11b | M1/70 | Biolegend | 1:200 |
| MHCII | M5.114.15.2 | eBioscience | 1:400 |
| CD3 | 17A2 | Biolegend | 1:200 |
| CD19 | 6D5 | Biolegend | 1:200 |
| Ly6G | 1A8 | Biolegend | 1:200 |
| CD115 | AFS98 | Biolegend | 1:200 |
| Ly6C | MK1.4 | Biolegend | 1:400 |
| Pan-CD45 | 30-F11 | Biolegend | 1:200 |
| CD11c | N418 | Biolegend | 1:200 |
| CD64 | X54-5/7.1 | Biolegend | 1:200 |
| Streptavidin | — | BD biosciences | 1:200 |
| LIVE/DEAD ® | — | Life Technologies | 1:100 |
| Fixable Aqua | | or Biolegend | |

### 3 Results

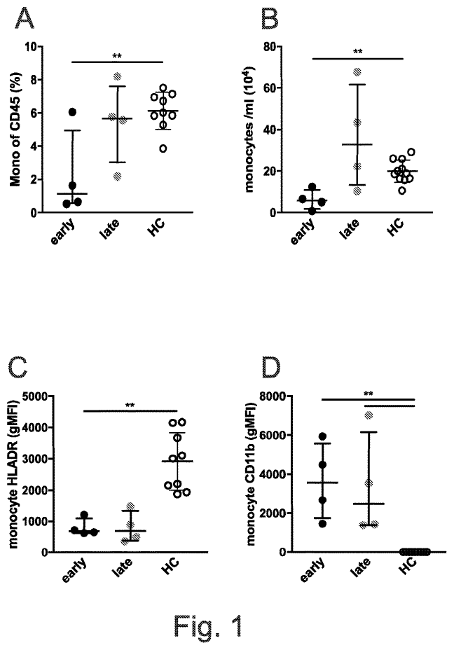

Flow cytometry analysis of blood samples from patients with ARDS and healthy donors, surprisingly revealed that patients with ARDS have a significant reduction in the proportion and number of circulating monocytes early in ARDS and a persistently altered monocyte phenotype compared to healthy controls (FIG. 1). Patients with ARDS have significantly less HLADR⁺ expression on their monocytes than heathy donors and significantly higher CD11b⁺ expression on their monocytes than heathy donors.

In an LPS mediated murine model of acute lung injury (ALI) and pneumonia, a reduction in circulating monocyte numbers is associated with a failure to induce CD64⁺Ly6C⁺ monocyte derived interstitial macrophages in the lung (FIGS. 2 and 3) and persistence of lung inflammation to day 5 (FIG. 3).

Treatment with CSF1 was found to rescue circulating monocyte and lung interstitial macrophage populations promoting inflammation resolution. Treatment with CSF1 increased both the proportion of circulating monocytes and cell count. Additionally, treatment with CSF1 increased lung monocyte cell count, interstitial macrophage cell count, and Ly6C⁺ interstitial macrophage cell count. Moreover, treatment with CSF1 reduced the proportion and numbers of lung neutrophils as well as bronchoalveolar (BAL) neutrophil numbers demonstrating accelerated lung inflammation resolution. As mice were treated with CSF1 not earlier than 24 from induction with LPS, this would have allowed sufficient time for neutrophil recruitment to take place prior to the provision of the treatment. According, a reduction on neutrophils is indicative of neutrophil clearance rather than mere prevention of neutrophil infiltration, rendering CSF1 useful as a therapeutic.

### Example 2

### Protocol for Prospective Monocyte Cell Therapy

Donor mice will be treated with nebulised LPS (3 mg) as described above in the methods section, to induce ALI, and will be housed in hypoxia (10%) at day 0. They will be then treated with CSF1 daily s.c. (0.75 ug/g) from day 1-4. Concurrently, recipient mice will be treated with nebulised LPS on day 4 and housed in hypoxia (10%). After treatment with LPS, suitably between day 5 and 10, the donor mice will be sacrificed, and blood collected. Blood monocytes will be sorted by flow cytometry-assisted cell sorting (FACS) (CD45+ CD11b+ CD3− CD19− Ly6G− CD115+ Ly6C+). Up to 1×10⁶ cells will be injected i.v. into the recipient mice in 100 μL PBS. Control mice will receive sham injections only. The recipient mice will be returned to hypoxia and at day 5, blood, bronchoalveolar lavage and lungs will be collected for measurements of inflammatory responses, including neutrophil counts. The timing of the cell therapy administration may be altered to include any timepoint from onset of ALI in the recipient mice. Determining optimal timing of cell therapy may be determined by the skilled person using routine experimentation.

### Example 3

### Bone Marrow Derived Macrophage Transfer

Bone marrow cells from wild-type mice were collected and flushed with media. The cells were then plated in low-adherence cell culture flasks and incubated for 7 days in standard culture conditions as explained hereinabove in relation to PBMC derived macrophages with media supplemented with mouse CSF-1 10 nanograms/ml. The media was changed every 3 days. The resultant cells were bone marrow-derived macrophages, and purity was determined using flow cytometry and assessing surface expression of F480 and CD11b. Recipient mice were treated with nebulised lipopolysaccharide (LPS) and placed in hypoxia (10%) to replicate the key characteristics of ARDS, hypoxia and acute lung injury.

After 24 hours, mice received a dose of 5 million BMDM cells or vehicle control intravenously, and then placed back into hypoxia for a further 24 hours.

Mice were sacrificed and lungs and bronchoalveolar (BAL) examined. Results are shown in FIG. 5.

### Example 4

Analysis of Phenotypic Switch in Blood Monocytes and Interstitial Lung Macrophages Treated with CSF1

Mice were treated with LPS and housed in normoxia (21% FiO2) or hypoxia (10% FiO2) for 5 days. Mice were treated with subcutaneous PBS or 0.75 micrograms/g CSF1 (hypoxia only) daily for 4 days. On day 5, mice were culled and blood collected. Classical monocytes were collected by using fluorescence-assisted cell sorting and samples run on the Nanostring platform as per manufacturer's instructions.

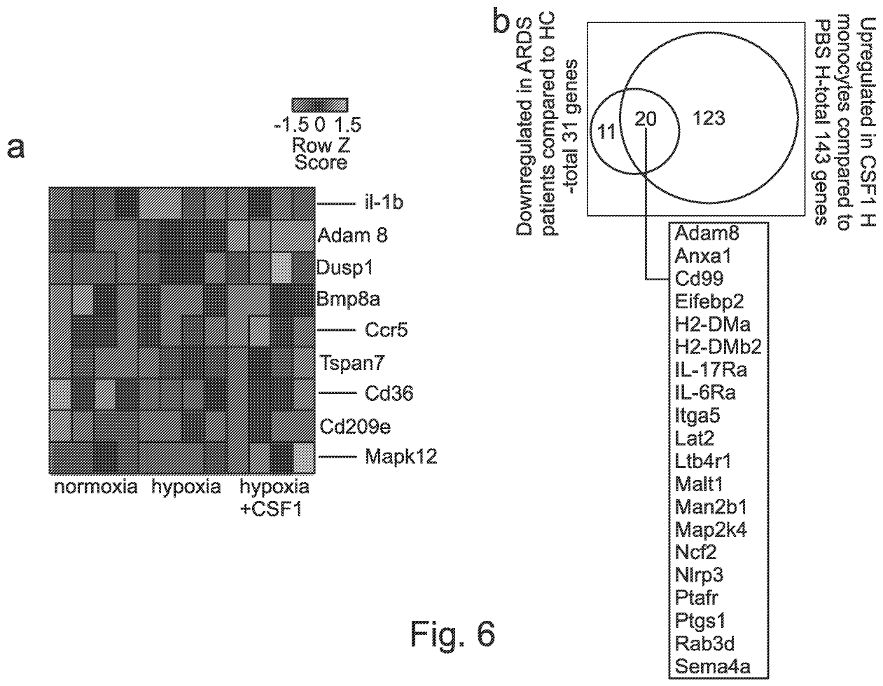

Differentially regulated genes identified by Nanostring were determined in these cells and results are shown in FIG. 6. The following genes were significantly upregulated in monocytes from CSF1 treated mice:

| | Log2FC | Lin FC |
|---|---|---|
| Adgre1 | 1.365 | 2.57576326 |
| Ccr5 | 1.35 | 2.54912125 |
| Ifnar1 | 0.7125 | 1.63864121 |
| Ifnar2 | 0.75 | 1.68179283 |
| Irf3 | 1.2225 | 2.33350733 |

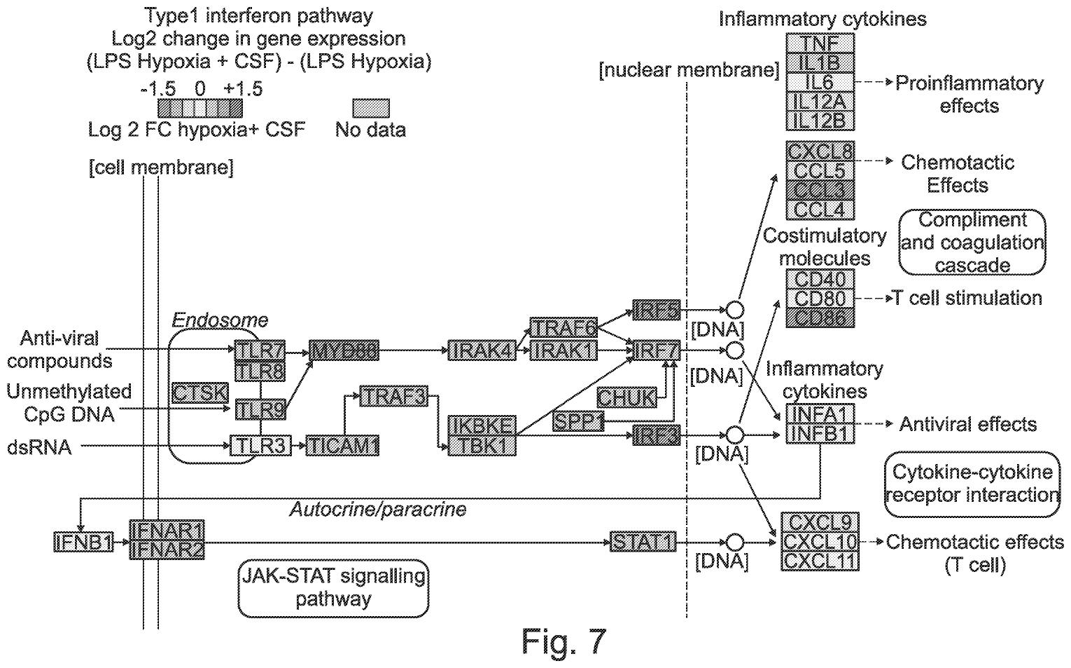

Kegg pathway analysis of the differentially expressed genes was performed and is shown in FIG. 7. FIGS. 6 and 7 show the increase in pro-restorative genes expressed by the monocytes from mice treated with CSF1.

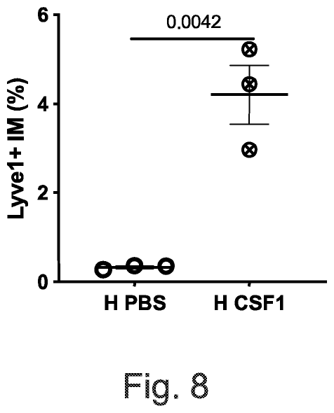

Lung digest was performed in mice treated with LPS and housed in hypoxia (FiO2 10%) for 5 days, treated daily with CSF1 0.75 micrograms/g for 4 days. The numbers of Lyve1+ lung interstitial macrophages were analysed in CSF1 treated hypoxic ALI mice compared to PBS treated hypoxic ALI mice. Lyve1+ macrophages have been shown to have a pro-restorative phenotype. Their numbers were increased in mice treated with CSF-1, see FIG. 8.

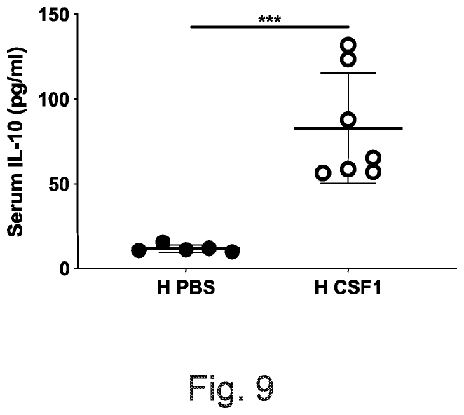

IL-10 concentration in serum was further measured in the mice treated with LPS and housed in hypoxia (FiO2 10%) for 5 days and treated daily with CSF1 0.75 micrograms/g for 4 days compared to the PBS treated hypoxic ALI mice. IL-10 in serum was increased in the mice treated with CSF-1, see FIG. 9.

### REFERENCES

Walmsley S R, et al. Hypoxia-induced neutrophil survival is mediated by HIF-1 alpha-dependent NF-kappaB activity. J Exp Med. 2005 Jan. 3; 201(1):105-15.
Eltzschig H K, Carmeliet P. Hypoxia and inflammation. N Engl J Med. 2011 Feb. 17; 364(7):656-65.
Zemans R L, Matthay M A. What drives neutrophils to the alveoli in ARDS. Thorax. 2017 January; 72(1):1-3.
Wang F, et al. Bone marrow derived M₂ macrophages protected against lipopolysaccharide-induced acute lung injury through inhibiting oxidative stress and inflammation by modulating neutrophils and T lymphocytes responses. Int Immunopharmacol. 2018 August; 61:162-168. doi: 10.1016/j.intimp.2018.05.015. Epub 2018 Jun. 5.
Gow D J, et al. Characterisation of a novel Fc conjugate of macrophage colony stimulating factor. Mol Ther. 2014 September; 22(9):1580-92. doi: 10.1038/mt.2014.112. Epub 2014 Jun. 25.

SEQUENCES
amino acid sequence of CSF1
SEQ ID NO: 1
MTAPGAAGRC PPTTWLGSLL LLVCLLASRS ITEEVSEYCS
HMIGSGHLQS LQRLIDSQME TSCQITFEFV DQEQLKDPVC
YLKKAFLLVQ DIMEDTMRFR DNTPNAIAIV QLQELSLRLK
SCFTKDYEEH DKACVRTFYE TPLQLLEKVK NVFNETKNLL
DKDWNIFSKN CNNSFAECSS QDVVTKPDCN CLYPKAIPSS
DPASVSPHQP LAPSMAPVAG LTWEDSEGTE GSSLLPGEQP
LHTVDPGSAK QRPPRSTCQS FEPPETPVVK DSTIGGSPQP
RPSVGAFNPG MEDILDSAMG TNWVPEEASG EASEIPVPQG
TELSPSRPGG GSMQTEPARP SNFLSASSPL PASAKGQQPA
DVTGTALPRV GPVRPTGQDW NHTPQKTDHP SALLRDPPEP
GSPRISSLRP QGLSNPSTLS AQPQLSRSHS SGSVLPLGEL
EGRRSTRDRR SPAEPEGGPA SEGAARPLPR FNSVPLTDTG
HERQSEGSFS PQLQESVFHL LVPSVILVLL AVGGLLFYRW
RRRSHQEPQR ADSPLEQPEG SPLTQDDRQV ELPV
amino acid sequence of an exemplary fragment of
CSF1
SEQ ID NO: 2
SENCSHMIGDGHLKVLQQLIDSQMETSCQIAFEFVDQEQLTDPVCYLKK
AFLQVQDILDETMRFRDNTPNANVIVQLQELSLRLNSCFTKDYEEQDKA
CVRTFYETPLQLLEKIKNVFNETKNLLKKDWNIFSKNCNNSFAKCSSQH
ERQPEGR
amino acid sequence of an exemplary antibody
fragment
SEQ ID NO: 3
GTKTKPPCPICPGCEVAGPSVFIFPPKPKDTLMISQTPEVTCVVVDVSK
EHAEVQFSWYVDGVEVHTAETRPKEEQFNSTYRVVSVLPIQHQDWLKGK
EFKCKVNNVDLPAPITRTISKAIGQSREPQVYTLPPPAEELSRSKVTVT
CLVIGFYPPDIHVEWKSNGQPEPEGNYRTTPPQQDVDGTFFLYSKLAVD
KARWDHGETFECAVMHEALHNHYTQKSISKTQGK
amino acid sequence of an exemplary fusion
protein HCSF1-MFC (CD33-HSCSF1 (AA36-190)-GS-
PX-GSS-MFC2A LALA)
SEQ ID NO: 4
MTAPGAAGRC PPTTWLGSLL LLVCLLASRS ITEEVSEYCS
HMIGSGHLQS LQRLIDSQME TSCQITFEFV DQEQLKDPVC
YLKKAFLLVQ DIMEDTMRFR DNTPNAIAIV QLQELSLRLK
SCFTKDYEEH DKACVRTFYE TPLQLLEKVK NVFNETKNLL
DKDWNIFSKN CNNSFASCSS QDVVTKPDCN CLYPKAIPSS
DPASVSPHQP LAPSMAPVAG LTWEDSEGTE GSSLLPGEQP
LHTVDPGSAK QRPPRSTCQS FEPPETPVVK DSTIGGSPQP
RPSVGAFNPG MEDILDSAMG TNWVPEEASG EASEIPVPQG
TELSPSRPGG GSMQTEPARP SNFLSASSPL PASAKGQQPA
DVTGTALPRV GPVRPTGQDW NHTPQKTDHP SALLRDPPEP
GSPRISSLRP QGLSNPSTLS AQPQLSRSHS SGSVLPLGEL
EGRRSTRDRR SPAEPEGGPA SEGAARPLPR FNSVPLTDTG
HERQSEGSFS PQLQESVFHL LVPSVILVLL AVGGLLFYRW
RRRSHQEPQR ADSPLEQPEG SPLTQDDRQV ELPV

1. An agent for increasing mononuclear cell number in a subject for use in the treatment of a lung disease associated with dysfunctional neutrophilic inflammation.

2. A method for treating a lung disease associated with dysfunctional neutrophilic inflammation in a subject, the method comprising providing the subject with a therapeutically effective amount of an agent for increasing mononuclear cell number in the subject.

3. Use of an agent for increasing mononuclear cell number in the manufacture of a medicament for the treatment of a lung disease associated with dysfunctional neutrophilic inflammation.

4. The agent for use, the method or the use according to any one of claims 1 to 3, wherein the agent increases monocyte and/or macrophage cell number in the lungs of the subject.

5. The agent for use, the method or the use according to claim 4, wherein the agent increases the number of pro-restorative monocyte cells and/or the number of pro-restorative macrophage cells in the lungs of the subject.

6. An agent for inducing a pro-restorative phenotype in mononuclear cells in a subject for use in the treatment of a lung disease associated with dysfunctional neutrophilic inflammation.

7. A method for treating a lung disease associated with dysfunctional neutrophilic inflammation in a subject, the method comprising providing the subject with a therapeutically effective amount of an agent for inducing a pro-restorative phenotype in mononuclear cells in the subject.

8. Use of an agent for inducing a pro-restorative phenotype in mononuclear cells in the manufacture of a medicament for the treatment of a lung disease associated with dysfunctional neutrophilic inflammation.

9. The agent for use, the method or the use according to any preceding claim, wherein the agent is selected from the group consisting of a polypeptide, a nucleic acid, a mononuclear cell, a small molecule, or any combination thereof.

10. The agent for use, the method or the use according to claim 9, wherein the polypeptide is selected from the group consisting of a cytokine, an enzyme, a hormone, a growth factor, a regulatory protein, and an immunoregulator, or a fragment, variant or homolog thereof.

11. The agent for use, the method or the use according to claim 10, wherein the cytokine is selected from the group consisting of CSF1 and IL-34.

12. The agent for use, the method or the use according to claim 11, wherein CSF1 has an amino acid sequence at least 75% identical to SEQ ID NO: 1.

13. The agent for use, the method or the use according to claim 9, wherein the nucleic acid is DNA or RNA.

14. The agent for use, the method or the use according to claim 9, wherein the mononuclear cell is a monocyte, a macrophage, or a monocyte precursor.

15. The agent for use, the method or the use according to claim 14, wherein the mononuclear cell has a pro-restorative phenotype.

16. The agent for use, the method or the use according to claim 14, wherein the monocyte:
has a phenotype characteristic of a circulatory monocyte or a tissue monocyte; and/or
has one or more markers selected from the group consisting of CD45⁺, HLADR⁺, CD14⁺, CD16⁺, CD11b⁺, CD206⁻, CD169⁻, and/or
is non-granulocytic.

17. The agent for use, the method or the use according to claim 14, wherein the macrophage is monocyte derived macrophage and/or a tissue macrophage.

18. The agent for use, the method or the use according to claim 17, wherein the monocyte derived macrophage and/or tissue macrophage is a lung macrophage, a bone-marrow derived macrophage, or a PBMC derived macrophage.

19. The agent for use, the method or the use according to claim 17, wherein the monocyte derived macrophage has one or more markers characteristic of an alveolar macrophage, optionally wherein the monocyte derived macrophage has one or more markers selected from the group consisting of CD11b⁺, HLADR⁺, CD206⁺, and CD169⁺.

20. The agent for use, the method or the use according to claim 17, wherein the monocyte derived macrophage has one or more markers characteristic of an interstitial macrophage, optionally wherein the monocyte derived macrophage has one or more markers selected from the group consisting of CD11b⁺, HLA-DR⁺, CD206⁺, and CD169⁻.

21. The agent for use, the method or the use according to claim 9, wherein the pharmaceutical is a small molecule.

22. A mononuclear cell having a pro-restorative phenotype for use in the treatment of a lung disease associated with dysfunctional neutrophilic inflammation.

23. A method for treating a lung disease associated with dysfunctional neutrophilic inflammation in a subject, the method comprising providing the subject with a therapeutically effective amount of a mononuclear cell having a pro-restorative phenotype.

24. Use of a mononuclear cell having a pro-restorative phenotype in the manufacture of a medicament for the treatment of a lung disease associated with dysfunctional neutrophilic inflammation.

25. A mononuclear cell having a pro-restorative phenotype for use, the method, or the use according to any of claims 22-24, wherein the cell has an upregulated type I interferon pathway.

26. A mononuclear cell having a pro-restorative phenotype for use, the method, or the use according to any of claims 22-26, wherein the cell has increased expression of one or more genes in the type I interferon pathway selected from: IRF3, IFNAR1, and IFNAR2.

27. A mononuclear cell having a pro-restorative phenotype for use, the method, or the use according to any of claims 22-27, wherein the cell has increased expression of one or more monocyte function genes selected from: ADGRE1, CCR5 and F480.

28. A mononuclear cell having a pro-restorative phenotype for use, the method, or the use according to claim 27, wherein the cell is a monocyte having a pro-restorative phenotype.

29. The agent for use, the method or the use according to any preceding claim wherein the lung disease associated with dysfunctional neutrophilic inflammation is selected from the group consisting of acute respiratory distress syndrome (ARDS), chronic obstructive pulmonary disease (COPD), and lung infection.

30. The agent for use, the method or the use according to any preceding claim, wherein the dysfunctional neutrophilic inflammation is in the lung.


SEQUENCE LISTING (4 sequences)

SEQ ID NO: 1 - protein, 554 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSFSPQLQESVFHLLVPSVILVLLAVGGLLFYRWRRRSHQEPQRADSPLEQPEGSPLTQDDRQVELPV

SEQ ID NO: 2 - protein, 154 aa, Artificial Sequence
  amino acid sequence of an exemplary fragment of CSF1
SENCSHMIGDGHLKVLQQLIDSQMETSCQIAFEFVDQEQLTDPVCYLKKAFLQVQDILDETMRFRDNTPNANVIVQLQELSLRLNSCFTKDYEEQDKACVRTFYETPLQLLEKIKNVFNETKNLLKKDWNIFSKNCNNSFAKCSSQHERQPEGR

SEQ ID NO: 3 - protein, 230 aa, Artificial Sequence
  amino acid sequence of an exemplary antibody fragment
GTKTKPPCPICPGCEVAGPSVFIFPPKPKDTLMISQTPEVTCVVVDVSKEHAEVQFSWYVDGVEVHTAETRPKEEQFNSTYRVVSVLPIQHQDWLKGKEFKCKVNNVDLPAPITRTISKAIGQSREPQVYTLPPPAEELSRSKVTVTCLVIGFYPPDIHVEWKSNGQPEPEGNYRTTPPQQDVDGTFFLYSKLAVDKARWDHGETFECAVMHEALHNHYTQKSISKTQGK

SEQ ID NO: 4 - protein, 554 aa, Artificial Sequence; identical to SEQ ID NO: 1
  amino acid sequence of an exemplary fusion protein HCSF1-MFC (CD33-HSCSF1 (AA36-190)-GS-PX-GSS-MFC2A LALA)